(19) United States
(12) Patent Application Publication
(10) Pub. No.: US 2009/0035260 A1
(43) Pub. Date: Feb. 5, 2009

(54) ENHANCED NASAL COMPOSITION OF ACTIVE PEPTIDE

(75) Inventors: Paolo Alberto Veronesi, Milan (IT); Pablo E.A. Rodriguez, Cordoba (AR)

Correspondence Address: STANDLEY LAW GROUP LLP, 6300 Riverside Drive, Dublin, OH, 43017, US

(73) Assignee: THERAPICON SRL, Milan (IT)

(21) Appl. No.: 12/186,251

(22) Filed: Aug. 5, 2008

Related U.S. Application Data
(63) Continuation-in-part of application No. 10/516,613, filed on Dec. 3, 2004, now abandoned.

(30) Foreign Application Priority Data
Jul. 29, 2002 (IT) MI2002A001684

(51) Int. Cl. A61K 38/20; A61K 38/21; A61K 38/19; A61K 39/00; A61K 38/49; A61K 38/44; A61K 38/16; A61P 3/10

(52) U.S. Cl. 424/85.2; 424/85.4; 424/85.1; 424/184.1; 424/94.63; 424/94.4; 514/12; 424/94.6

(57) ABSTRACT

A pharmaceutical composition has a therapeutically effective amount of at least one of: a pharmaceutically active nasal peptide, its pharmaceutically acceptable salt and its peptidic fragment. The composition also contains an absorbefacient effective amount of THAM in a pharmaceutically acceptable, aqueous liquid diluent or carrier. The composition is provided in a convenient form for nasal administration. In one embodiment, the peptidic fragment may be selected physiologically active lymphokines and monokines, peptidic enzymes, proteic vaccines, peptidic toxoids and personalized proteins derived from genoma. In another embodiment, the peptidic fragment may be selected from the peptide hormones and hormone antagonists buserelin, desmopressin, vasopressin, angiotensin, felypressin, octreotide, somatropin, thyrotropin (TSH), somatostatin, gosereline, thryptorelin and insulin selected from the group consisting of cow and pig, synthetic and recombinant.

### CROSS-REFERENCE TO RELATED APPLICATIONS

This application is a continuation-in-part of U.S. Ser. No. 10/516,613, filed 3 Dec. 2004, which is a national stage entry under 35 USC §371 of PCT/EP03/06641, filed 24 Jun. 2003, which claims a right of priority under 35 USC §119 from Italian patent application M12002A 001684, filed 29 Jul. 2002. Each of these applications is incorporated by reference as if fully recited herein.

### TECHNICAL FIELD

This invention relates to an enhanced pharmaceutical composition comprising a combination of a therapeutically active ingredient selected from a natural, synthetic or recombinant peptide or a polypeptide or any of its pharmaceutically acceptable salts or peptidic fragments thereof, or a personalized peptide or a mixture thereof (hereinafter, for the sake of convenience, defined “nasal peptide”) and of an absorbefacient effective amount of tris (hydroxymethyl) amino methane (THAM) in a ready-to-use aqueous solution designed for the nasal administration of said nasal peptide. The present invention also relates to a method for producing the ready-to-use solution of the pharmaceutical composition containing the nasal peptide, which can be put up in a mono-disposable or in a multidose delivery system device suitable for the intranasal administration route.

### BACKGROUND ART

Pharmacologically active peptides, such as buserelin, desmopressin, vasopressin, octreotide and many other medium and long-chain peptides or polypeptides in current therapeutic use are easily degraded by the enzymes in the human stomach and intestine or are easily metabolized in the liver. Therefore, such peptides and polypeptides cannot be administered by the oral route, because they are not absorbed or are absorbed with difficulty through the gastrointestinal tract and therefore cannot elicit their systemic pharmaceutical effects in patient's body.

Thus, heretofore, the administration route for such peptides, has been by virtue of hypodermic, intramuscular or intravenous injections. However, the patient often experiences pain and irritation at the injection site, mainly due to injury or tissue necrosis during long-term treatments. In addition, injections potentially present the risk of secondary infections due to communicable diseases.

Even the most recent injectable compositions designed for long-term administration (weekly or monthly doses) of peptides (U.S. Pat. No. 5,582,591; U.S. Pat. No. 5,776,885 and U.S. Pat. No. 6,376,461) do not satisfactorily solve patient's compliance. However, the liberation rate of the peptide presents an high peak immediately after injection (the release of the peptide is not as gradual as required), the residual quantity of chlorate organic solvents used for their production is too high and risky for the patient's health and often the final product containing the peptide can be sterilized only by gamma-radiation, with the consequent possible health risks. However, the above inconveniences represent only part of the unsolved technical problems still faced by patients when using long-lasting (depot) preparations for parenteral administration containing a peptide or polypeptide.

Other alternative methods for the administration of peptides have been tentatively proposed in the past, such as, for instance, the rectal administration of a suppository (J. Pharm. Pharmacol., 33, 334, 1981), the endotracheal administration (Diabetes, 20, 552, 1971) and also eyedropping administration (J. Diabetic Society, Summary, 237, 1974). However, none of these attempts have entered into current clinical practice because of the unsatisfactory absorption rate, the great variation in absorption and of the secondary irritation caused by absorption enhancers, preservation agents and auxiliary ingredients.

During the last decade several authors have performed multiple attempts to develop compositions intended to deliver peptides through the nasal mucosa. Nevertheless, most of the technical solutions proposed in the past have not produced the desired results. The compositions used unexpectedly presented stability problems and even caused side effects so that only a few products were marketed and with poor commercial results.

However, peptides are remarkably unstable in aqueous solutions and easily degrade with concomitant loss of activity and simultaneous accumulation of undesired degradation products. Therefore, some authors have tried to develop dry powder compositions for nasal administration (EP0302772; EP 0468182 and WO99/59543, WO2007/126865), which are chemically more stable than the liquid compositions. However the dry powder compositions present other inconveniences related to patient's compliance, so that these solutions have been rapidly abandoned.

Other attempts to obtain stable aqueous solutions to be used for the nasal administration of pharmaceutically active peptides have been rather limited and often are only applicable to a specific peptide, as for example the nasal administration of insulin (EP 94157), vasopressin (EP55066-517; JP55055-120) and of other peptides (DE 2.256.445; DE 2.758.463; BE 860.717). In addition, most of the absorption enhancers, preserving agents or auxiliary ingredients of the compositions may be stability destroying factors so that it is rather difficult to combine them in a convenient and stable liquid composition containing a peptide. Any published solutions are suitable only for the specific peptide for which they have been designed, so that the same technical solution cannot generally be applied to other nasal peptides.

Therefore, there is still an urgent need for a general pharmaceutical composition which allows one to conveniently formulate a large number of peptides or polypeptides to be administered by intranasal route. However, once the technical problems of stability of the composition containing the nasal peptide have been solved, thereafter there are other problems that may unexpectedly appear such as undesirable side effects caused by absorption enhancers and/or preserving agents and/or auxiliary excipients and/or metals that may clinically produce undesirable adverse effects of serious intensity.

An example is the widespread use of benzalkonium chloride as absorption enhancer and/or as preserving agent, despite the fact that it produces undesirable side effects (Am. J. Rhinol., 22(2), p. 125-9, 2008; Invest. Opthalmol. Vis. Sci., 49(5), p. 1850-6, 2008; Am. J. Rhinol., 18(5), p. 291-9, 2004; Br. J. Opthalmol., 91(11), p. 1460-3, 2007; Invest. Opthalmol. Vis. Sci., 46(2), p. 703-8, 2005; Amer. J. Ophtalmol. 105(6), p. 670-73, 1988; Contact Dermatitis, 53(2), p. 93-9, 2005; Contact Dermatitis, 17(1), p. 41-2, 1987; Cutis, 39(5), p. 381-83, 1987; Mutagenesis, 22(6), p. 363-70, 2007; Toxicol. In Vitro., 20(8), p. 1472-7, 2006, to name a few.

Another typical case of intolerance arises due to the presence of the preserving agent chlorobutanol, which may cause sensibilization problems when used for long term treatments involving the nostrils, as reported in the literature (Ann. Pharmacother., 30(10), p. 1179-80, 1996; Acta Otolaryng., 70, p. 16-26, 1970; Ophtalmol. Vis. Sci., 71(9), p. 562-72, 1994; Teratology, 47(3), p. 203-8, 1993; Lens Eye Toxic. Res., 6(3), p. 395-403, 1989; Med. J. Aust., 1(7), p. 288, 1979; Merck Index Twelfth Edition entry n. 2148; U.S. Pat. No. 5,759,565).

Furthermore, the intranasal administration of zinc or its salts represents another case of nasal intolerance to current ingredients used for the preparations of pharmaceutical compositions for intranasal administration of peptides. Zinc is an essential element, which can act as a neuromodulator and also is bound in zinc proteins in the brain. Many nasal preparations for cold or flu contain zinc salts or complexes because of the strong antiviral activity that has been attributed to this essential element (U.S. Pat. No. 7,348,360; US 2008085298; US 2007265337; MXPA 05013214; U.S. Pat. No. 6,638,915; U.S. Pat. No. 6,500,808).

Zinc salts are also used as stabilizers and release modifiers for insulin, and bovine and porcine insulin have been modified by the addition of a suitable zinc salt (U.S. Pat. No. 6,734,162 to Minimed Inc.). By contrast, it has been recently found that the inhalation of zinc containing powder, dust or fumes may cause neurotoxicity in the olfactory system and this is an issue which deserves attention, since zinc dysregulation has been implied to pay a role in Alzheimer's disease. In addition, impairment of the sense of smell and degenerative changes of the olfactory tissues have been seen in early stages of some neurodegenerative disorders (Toxicology, 191, 2003, p. 97-108; Microsc. Res. Tech., 1993, 24 (3), p. 195-213; Chem. Senses, 28(8), p. 659-70, 2003; Laryngoscope, 117(4), p. 743-9, 2007; Am. J. Rhinol., 18(3), p. 137-41, 2004).

The consequence is that zinc containing substances, such as zinc insulin, can be used only theoretically by nasal administration, since the presence of zinc in the nasal composition may seriously affect patient's compliance when administered by nasal route.

On the other hand, the auxiliary ingredient THAM, which is known in literature under the synonyms tromethane, tromethamol, TRIS or under the chemical name [tris (hydroxymethyl) aminomethane] is an alkalinizing, buffering and pH correcting agent used in many pharmaceutical compositions (U.S. Pat. No. 4,001,327; U.S. Pat. No. 4,368,193; U.S. Pat. No. 4,423,067; U.S. Pat. No. 4,464,466; U.S. Pat. No. 5,080,906; U.S. Pat. No. 6,577,947; U.S. Pat. No. 7,022,687; U.S. Pat. No. 7,045,323), including some nasal compositions.

The selection and the combination in a nasally administrable pharmaceutical composition of a peptide and of an absorption enhancer and/or preserving agent(s) and/or auxiliary excipients(s) and/or accessory metal(s) would not be immediately obvious for a skilled artisan, in view of the fact that it would not be easy to differently combine teachings and/or published literature in order to yield a composition showing all desired properties. In fact, very often the inclusion of an ingredient to overcome or to correct an existing problem, may seriously affect or even destroy another essential characteristic of the peptide itself or of the pharmaceutical composition.

In light of this there has not yet been developed an enhanced nasally administrable pharmaceutical composition including such a peptide and combining such desired properties, i.e. to be an aqueous composition conveniently applicable to a large number of therapeutically active peptides, that can be administered by nasal route, and which can achieve a constant absorption rate and a therapeutically effective amount of peptide to elicit the desired systemic effect, and also simultaneously overcoming the stability problems typically of a peptide in aqueous solution without inducing adverse side effects such as those previously described herein.

In addition, the capacity of the human nasal cavity surface for holding aqueous solutions of most nasally administered agents is less than 400 microliters, while, on the other hand, about 100 microliters is the lowest dosing volume that can be conveniently reproduced by single actuations of the metered device. However, for an efficient systemic absorption of nasally administered therapeutics, the vehicle carrying the drug must remain in contact with the mucus-lined epithelium for a sufficient period of time.

Therefore, it has previously been proposed (EP 0122036) to include in the composition viscosity modifying agents such as methylcellulose, crospovidone or povidone to try and prolong the contact time of the preparation with the nasal epithelium. Nevertheless, a publication of experimental studies in rabbits clearly shows that the increased viscosity negatively influences the intranasal absorption of salmon calcitonin.

Similarly, while many skilled persons in the art believe that isotonicity is a positive factor for the intranasally administered peptide, on the contrary, a study (Int. Jour. of Pharm., 147, p 233-242, 1997) showed the negative influence of tonicity on the intranasal absorption of a peptide. Therefore, isotonic solutions are to be avoided in order to optimize the intranasal absorption of the desired peptide.

Despite the fact that nasal administration of active peptides is described in the literature, different authors have reported either limited achievements and partially satisfactory or contradictory findings: Experientia, 25(11), p. 1195-6, 1969; Lancet, 1(7862), p. 865, 1974; Antimicrobial Agents Chemother., 14(4), p. 596-600, 1978; Lancet, 2(8136), p. 215-7, 1979; Br. Med. J., 284(6312), p. 303-6, 1982; Aerosols in Medicine, Elsevier Scientific, 1983, p, 346; WO2004084859; WO2004224890; U.S. Pat. No. 5,733,569; U.S. Pat. No. 5,759,565; EP0726075 (U.S. Pat. No. 6,087,338); U.S. Pat. No. 6,734,162; U.S. Pat. No. 6,440,392; EP0302772.

Among those proposed solutions, EP0726075 (U.S. Pat. No. 6,087,338) discloses pharmaceutical non-inorganic saline solutions for endonasal administration containing a peptide (a calcitonin), meglumine or glucamine, citric acid, polyvinylpyrrolidone and tromethane (THAM): Such a solution is disclosed to overcome the problem of the remarkable instability of calcitonins. Throughout this prior art, the presence of tromethane is described to provide a partial buffering agent for the citric acid, in particular to maintain the pH at 4.6 to 6.0. Nowhere is there any suggestion that tromethane (THAM) is capable of exhibiting enhanced absorbefacient properties.

U.S. Pat. No. 6,734,162 (WO 01/52937) discloses that a mixed TRIS (THAM) buffer system provides improvements in the stabilization of polypeptides by inhibiting the aggregation of polypeptide pharmaceuticals. Specifically TRIS (THAM) supplies the negative coefficient of pH in combination with other buffering molecules. However, there is no teaching or disclosure anywhere that TRIS (THAM) is a useful absorbefacient material useful to increase the absorption of pharmaceutical compositions.

Another technical aspect practically ignored in literature, but which is well known to a skilled person in the art is that peptides formulated in aqueous solutions are rather unstable. The preferred storage conditions for peptides are: the physical solid state, storage temperature around or below 0° C. and accurate protection from oxygen, which is mainly responsible for the degradation processes involving the oxidation of the disulphide bonds and/or aminic radicals, which characterize the three-dimensional structure of the peptides and their biological activity. Therefore it is essential to ensure the above storage conditions and to avoid or limit as much as possible the accidental contact with oxygen.

Similarly, equivalent conditions shall be conveniently observed for peptides which are formulated in pharmaceutical compositions. However, it has been observed that isotonicity and the pH range of the solution containing the peptide do not appear to be essential conditions either for enhancing the absorption of the peptides nor to ensure the stability of the compositions.

Another desired effect is to stabilize the pharmaceutical compositions comprising the therapeutic peptide so as to protect it from oxygen and it is well known that the use of nitrogen may protect from oxidation. Moreover, the stability requirement for such a pharmaceutical composition containing the peptide shall not be limited only to the storage shelf life before its use, but it shall be extended also to the in-use stability after opening, particularly when a multidose container is used, as recommended by recent note for guidance adopted by some regulatory authorities, as for example The European Agency for the Evaluation of Medicinal Products (EMEA)-Note for Guidance CPMP/QWP/2934/99, September 2001, available at hppt:/www.emea.eu.int/. In other words, the technical preparation of an enhanced pharmaceutical compositions of a peptide intended to be administered by nasal route to yield a therapeutically effective level to exert a systemic effect it not a simple exercise, since it is necessary to simultaneously overcome several stringent problems.

### OBJECTS OF THE INVENTION

Thus, the problem underlying the present invention is to create an enhanced nasally administrable pharmaceutical composition suitable for delivering by the nasal route any natural, synthetic or recombinant peptide or polypeptide or any of its pharmaceutically acceptable salts or peptidic fragments thereof, or a personalized peptide or a mixture thereof, the pharmaceutical composition comprising a convenient combination of a selected effective amount of nasal peptide and of an absorbefacient effective amount of THAM, in order to achieve constant absorption rates and therapeutically effective levels of peptide to elicit the desired systemic effect and to improve the patient's compliance. Another benefit of the present invention is to preserve the nasal peptide from the stability problems affecting those compositions throughout its storage shelf life time. A further aspect underlying the present invention is a method for producing the pharmaceutical composition of the invention as ready-to-use solution, which may be conveniently put up in a mono-disposable or in a multidose delivery system device suitable for the intranasal administration route.

### DESCRIPTION OF THE INVENTION

Surprisingly this has been attained by the present invention. This invention is based on the unexpected recognition that a pharmaceutical composition for nasal administration of any natural, synthetic or recombinant origin peptide or a polypeptide or any of its pharmaceutically acceptable salts or peptidic fragments thereof, or a personalized peptide or a mixture thereof, may conveniently comprise an aqueous solution of an affective amount of the selected nasal peptide and of an absorbefacient effective amount of THAM, optionally comprising other pharmaceutically acceptable auxiliary additives and/or carriers, such a formulation advantageously overcomes the stability problems typically reported for peptides formulated in aqueous solutions.

The pharmaceutical composition of the instant invention exhibits enhanced absorption rates of the peptide to elicit the therapeutically systemic effect expected from the selected peptide, while concomitantly overcoming the stability problems typical of a peptide dissolved in aqueous solution, without inducing adverse reactions affecting the patient's compliance, typically associated with the use of unsuitable ingredients typical of other nasal compositions.

Another embodiment of the instant invention is that the pharmaceutical composition may be conveniently included in a mono-disposable or in a multidose delivery system device, both suitable for the intranasal administration route.

In another embodiment the absorbefacient effective amount of THAM has been purposely selected, THAM being the only hydrogen-ion acceptor amine without any marked toxicity (most amines produce marked toxic effects in vivo when used in dose sufficient quantities). It has also been previously found that the absorbefacient amount of THAM is convenient to physiologically and reversibly depolarize the monolayer epithelial cells of the nasal mucosa. However, at the conveniently selected amount THAM elicits its absorbefacient activity by enhancing the permeability and improving the efficiency of active absorption of the peptide through the nasal mucosa.

Therefore, the instant invention discloses using an absorbefacient effective amount of the amine THAM which significantly characterizes the pharmaceutical composition of the instant invention containing the nasal peptide to be administered and dosed by nasal route.

However, the present inventors have surprisingly found that the absorbefacient amount of THAM may be used, depending on the peptide to be included in the composition, in a concentration of 4.5 mg/ml up tol0.0 mg/ml. Furthermore, including THAM in an amount above 10 mg/ml does not exhibit any proportional absorbefacient effect.

It has also been unexpectedly found that the pharmaceutical composition of the invention is remarkably suitable for incorporating individually or in association, any natural, synthetic or recombinant peptide or polypeptide or its pharmaceutically acceptable salts or peptidic fragments thereof, or any personalized peptide or a mixture thereof, and which can be conveniently administered by intranasal route to elicit the systemic effect therapeutically expected from the individually selected or associated peptide. In fact, when applied to the mucus-lined epithelium as ready-to-use solution, the pharmaceutical composition can exhibit the desired pharmacological effect, as much as necessary to elicit the desired therapeutic activity in relation to the administration through the other applicable routes.

Thus in a first aspect, according to the present invention, the nasally administrable pharmaceutical composition comprises:

a therapeutically effective amount of one or more pharmaceutically active nasal peptides, or fragments or pharmaceutically acceptable salts thereof, and

an absorbefacient effective amount of THAM dissolved in a pharmaceutically acceptable aqueous carrier suitable for application to the mucus-lined epithelium of the nasal mucosa, said aqueous carrier comprising optionally other pharmaceutically acceptable auxiliary additives, such as (a) an inorganic or organic acid or a mixture thereof; (b) one or more preserving agents or a mixture thereof; (c) an amino acid co-formulator.

Therefore, the composition of the invention excludes viscosity modifying agents such as methylcellulose, crospovidone or povidone because of the adverse effect on the intranasal absorption of a peptide and also for their possible negative effect on the long term stability of the composition, with formation of opalescent micelles or precipitating agglomerates. Similarly isotonicity has been purposely avoided, since isotonic solutions seem to negatively influence the intranasal absorption of peptides.

In a further embodiment, the nasal peptide which can be advantageously administered according to the instant invention, are such peptides with a molecular weight ranging from 1000 to 150000 Dalton, in view of the fact that they are easily absorbed through the nasal mucous membrane. Especially those having a molecular weight ranging from 1000 to 50000 Dalton are more desirable. Such desirable physiologically active nasal peptides, which include also their pharmaceutically acceptable salts and their peptidic fragments, are exemplified in the following non-exhaustive list, which shall not be considered limiting.

For instance, such peptidic hormones and hormone derivatives as buserelin, desmopressin, vasopressin, angiotensin, felypressin, octreotide, somatropin, thyrotropin (TSH), somatostatin, gosereline, thryptorelin, insulin (obtained from caw and pig or synthetic or human recombinant but purposely not combined with zinc or other metals), protirelin, adrenocorticotropin (ACTH), prolactin, luteinizing hormone (LH), luteining hormone-release hormone (LH-RH), leuprorelin, calcitonin (human, chicken, eel, porcine or recombinant), carbocalcitonin, calcitonin gene related peptides (CGRP), kallikrein, parathyrin, glucagon, oxytocin, gastrin, secretin, leptin, nafarelin, serum gonadotropin, gonadotropin release factor, growth hormone, erytropoietin, hirudin, urograstrone, renin, human parathyroid hormone (h-PTH), teriparatide [h-PTH(1-34)]; such physiologically active proteins as lymphokine or monokine such as interferon and interleukin, transferrin, histaglobulin, macrocortine, endorphins, enkephalins, neurotensin; such as peptidic enzymes as lysozyme, urokinase, superoxide dismutase; such proteic vaccines as acellular and cellular pertussis vaccine, diphteria vaccine, tetanus vaccine, influenza vaccine; and such as peptidic toxoids as diphtheria toxoid, tetanus toxoid; can be suitably comprised in the pharmaceutical composition of the invention.

Personalised proteins, a new category of medicinal product of peptidic nature derived from genoma, which can be personalized for each patient for a specific disease, can be also conveniently included.

In one embodiment according to this invention, the therapeutically effective amount of nasal peptide and the absorbefacient effective amount of THAM are conveniently combined in an aqueous solution together with hydrochloric acid or citric acid or a mixture thereof and with one or a mixture of methyl or/and propyl p-hydroxybenzoate.

In another embodiment of the instant invention a nasally administrable pharmaceutical composition comprises a combination of:

a therapeutically effective amount of a pharmaceutically active nasal peptide from 0.001 mg/ml to 50.0 mg/ml or from 10 Units/ml to 20000 Units/ml, which may conveniently vary for each selected nasal peptide according to the unitary therapeutic dose to be applied by intranasal route; and

an absorbefacient effective amount of THAM from 4.5 mg/ml to 30.0 mg/ml, which may conveniently vary for each selected nasal peptide in relation to the required absorbefacient activity.

The pharmaceutically acceptable aqueous diluent or carrier may optionally comprise other pharmaceutically acceptable auxiliary additives, such as hydrochloric acid, preferably 0.1 N, from 0.3 mg/ml to 30.0 mg/ml or citric acid from 0.6 mg/ml to 60.0 mg/ml or a combined mixture thereof, and one or a mixture of methyl or/and propyl p-hydroxybenzoate from 0.1 mg/ml to 3.0 mg/ml.

In a particularly preferred embodiment of the instant invention the therapeutically effective amount of nasal peptide is from of 0.01 mg/ml to 10.0 mg/ml or from 20 Units/ml to 12500 Units/ml and the absorbefacient effective mount of THAM is in an amount from 4.5 mg/ml to 10.0 mg/ml, while the pharmaceutically acceptable aqueous carrier optionally further comprises citric acid monohydrate from 2.8 mg/ml to 6.2 mg/ml and a mixture of methyl and propyl p-hydroxybenzoate not exceeding 0.3 mg/ml in total, but with a ratio of 2:1 to 20:1.

A further surprising advantage of the instant invention is that THAM also contributes to the stabilization of the nasal peptide comprised in the pharmaceutical composition. In fact, it has been observed that liquid pharmaceutical compositions containing THAM do not easily absorb O2 and CO2 from the atmosphere, thus avoiding the contact with oxygen and improving the stability profile during production, storage and use, so that the production under nitrogen flow is also an optional choice.

A suitable nasal delivery system device used for dispensing the pharmaceutical composition of the invention is already available on the market and may dispense a single nasal dose unit or several sequential unit doses (multidose). The delivered quantity (metered volume) corresponds to a therapeutically effective dose unit of nasal peptide to be applied to the mucus-lined epithelium of the nasal cavity to obtain the therapeutic effect expected from the selected peptide. The container, the metered precision pump and the nasal applicator may be integrated also as a unit just dispensing one dose only and it can also be made disposable.

The delivery system device of a multidose dispenser may be also equipped with dose counting system.

The therapeutically effective dose of the nasal peptide to be contained in the pharmaceutical composition for intranasal administration may vary basically with the kind of selected nasal peptide and also with patient's age, body weight, severity of disease, desired therapeutic response, health conditions and other drugs simultaneously administered. Generally, the dose of the pharmaceutical composition for the nasal administration of the present invention which contains a pharmaceutically active nasal peptide may be determined according to the known administered doses of the selectively used nasal peptide.

The invention will now be explained in detail by reference to the following experimental examples, which are described only for presentation of further details rather than for limiting the scope of the invention itself.

### EXAMPLE 1

### Pharmaceutical Composition Containing Teriparatide [h-PTH (1-34)] Suitable for Nasal Administration and its Method of Preparation

A nasal spray pharmaceutical composition of teriparatide [human-PTH(1-34)] (1000 Units=1.0 mg/source: Martindale) having the following composition:

1) Teriparatide [h-PTH (1-34)] 1.0 mg (1000 Units)

### 2) THAM 4.60 mg

3) citric acid 6.60 mg
4) methyl p-hydroxybenzoate 2.50 mg
5) propyl p-hydroxybenzoate 0.30 mg
6) distilled water q.s. to 1.00 ml
was prepared as ready-to-use solution.

Ingredients were used in a scale volume to produce a final volume of 1.0 litre (corresponding to about 300 bottles of 3.0 ml each).

Ingredients 4) and 5) were dissolved in an aliquot of 800.0 ml of 6) and, after complete dissolution, ingredients 2) and 3) were added and mixed thoroughly. Finally, 1) was added to complete dissolution and the remaining 200.0 ml of 6) were added to yield 1.0 litre solution. The resulting solution was filtered by means of a 0.2 micron filter (Pall brand) to yield a composition suitable for nasal application. The filtered solution was used for filling, in a bacteriologically controlled area class 100 or 1000, into nasal spray multidose dispensers with a solution volume of 3.0 ml. Each container comprises 3.0 mg (3000 Units) of teriparatide [h-PTH(1-34)]/3.0 ml of solution and the metered pump system was suitable to deliver subsequent individual doses of 100 micrograms (100 Units) of teriparatide [h-PTH(1-34)]/100 microliters volume per each actuation.

Similarly, by using the same composition, metered dosing system and production techniques, but using half declared amount (0.5 mg=500 Units) and one quarter (0.25 mg=250 Units) of the ingredient 1), it was possible to obtain a ready-to-use solution delivering 50 micrograms (50 Units) and 25 micrograms (25 Units) of teriparatide [h-PTH(1-34)]/100 microliters volume of each actuation respectively.

In the same way, by using the same composition, metered dosing system and production techniques, but using double amount (2.0 mg=2000 Units) and four times (4.0 mg=4000 Units) of the ingredient 1), it was possible to obtain a ready-to-use solution delivering 200 micrograms (200 Units) and 400 micrograms (400 Units) of teriparatide [h-PTH(1-34)]/100 microliters volume of each actuation respectively.

### EXAMPLE 2

### Preliminary Report on the Enhanced Absorbefacient Effect Of THAM on Nasal Peptide Pharmaceutical Compositions (Vivo Studies)

### Introduction

Several scientific publications have recently reported an increasing interest for the administration of pharmaceutical compositions containing peptide compounds through the nasal mucosa. Due to the limited absorption rate of peptides through this administration route, the main interest of scientists during the most recent years was to enhance the absorption rate of the drug product by adopting a suitable absorbefacient agent. Nevertheless, published documents have neither considered nor reported any influence of enhancer concentration on the drug absorption profile.

### Scope

A study was therefore designed to support the absorbefacient effect of THAM in pharmaceutical nasal compositions containing known peptides, such as desmopressin and insulin. The study has been divided in two parts, one related to desmopressin nasal composition and the second to insulin nasal preparation.

### Desmopressin

### Materials

The following desmopressin nasal spray preparations were investigated and used as testing material, as shown in Table n. 1.

TABLE n. 1
Quali-quantitative compositions of Nasal Formulations of
desmopressin nasal spray solution to be tested.

| 1.0 ml of nasal spray solution | NASAL COMPOSITION N^{o}. | | | | | |
|---|---|---|---|---|---|---|
| containing: | 1 | 2 | 3 | 4 | 5 | 6 |
| Peptide and Ingredients: | mg | mg | mg | mg | mg | mg |
| Desmopressin acetate | 0.1126 | | | | | |
| (DDAVP) | (0.1000) | | | | | |
| (equivalent to desmopressin) | | | | | | |
| THAM | 1.0 | 2.0 | 4.50 | 10.0 | 20.0 | 30.0 |

| | |
|---|---|
| Citric acid | 6.28 |
| Methyl p-hydroxybenzoate | 0.27 |
| Propyl p-hydroxybenzoate | 0.03 |
| Distilled water q.s. to ml | 1.00 |

### Subjects and Methods

Administered single dose of 20 mg (2×10 mg) desmopressin, study design (4 healthy subjects), blood sampling and analytical procedures were carried out as described in the published literature (1). Each desmopressin Nasal Composition (as spray solution) was individually administered by intranasal administration route by means of a device (dosing pump and nasal applicator), such as those commonly available on the market. Each actuation volume dispensing 0.10 ml of nasal solution, corresponding to 20 mg desmopressin.

### Pharmacodynamic and Pharmacokinetic Calculations

For each tested Nasal Composition from No. 1 to 6, a blood sample was collected at 45 minutes after a single dose (20 mg) administration by nasal rout, being the Maximum level of desmopressin plasma concentration reached in about 45 minutes, as described in the published literature (1).

The individual desmopressin Maximum plasma concentrations and the mean (arithmetic mean and S.D.) plasma desmopressin concentrations are reported in Table n. 2.

TABLE n. 2
Individual desmopressin Maximum plasma concentrations and
arithmetic mean and S.D.

| Desmopressin Maximum | | | | | | |
|---|---|---|---|---|---|---|
| plasma concentrations | NASAL COMPOSITION N^{o}. | | | | | |
| (pg/ml) | 1 | 2 | 3 | 4 | 5 | 6 |
| Subject 1 | 23.3 | 28.4 | 45.7 | 47.3 | 31.5 | 29.3 |
| Subject 2 | 13.9 | 17.8 | 32.3 | 36.7 | 41.4 | 41.8 |
| Subject 3 | 25.1 | 16.8 | 44.1 | 47.9 | 29.7 | 29.8 |
| Subject 4 | 12.6 | 26.5 | 34.4 | 36.6 | 38.9 | 38.5 |
| Mean | 18.7 | 22.4 | 39.1 | 42.1 | 35.4 | 34.8 |
| S.D.± | 6.39 | 5.93 | 6.75 | 6.33 | 5.66 | 6.27 |

The significant absorbefacient effect of THAM in the tested Nasal Compositions is shown by the arithmetic mean Maximum plasma desmopressin concentrations (pg/ml) at 45 minutes following to a single dose nasal administration (20 mg) of each Nasal Composition and the results are reported in Table n. 3.

TABLE n. 3
Arithmetic mean of Maximum plasma desmopressin concentrations
(pg/ml).

| | NASAL COMPOSITION N^{o}. | | | | | |
|---|---|---|---|---|---|---|
| PARAMETERS | 1 | 2 | 3 | 4 | 5 | 6 |
| THAM concentrations | 1.0 | 2.0 | 4.5 | 10.0 | 20.0 | 30.0 |
| (mg/ml) | | | | | | |
| Arithmetic mean of | 18.7 | 22.4 | 39.1 | 42.1 | 35.4 | 34.8 |
| Desmopressin Maximum | | | | | | |
| plasma concentrations | | | | | | |
| (pg/ml) | | | | | | |

### Discussion and Conclusion

In this study the absorbefacient effect of THAM in pharmaceutical Nasal Compositions containing desmopressin has been investigated in relation to the absorption rate of the peptide product.

The experimental conditions and determinations of the case study have been carried out by partially following the applicable procedures, such as those described in published literature (1). The Maximum desmopressin plasma concentrations (pg/ml) have been determined at the predictable maximum peak of 45 minutes following to a single dose nasal administration of 20 mg of each Nasal Composition under testing. The results of the above study, clearly demonstrate that THAM exhibits an enhanced absorbefacient effect on the peptide desmopressin contained in Nasal Compositions.

### Insulin (Human Recombinant)

### Materials

Insulin (human recombinant) nasal spray preparations were investigated and used as testing material, as shown in Table n. 4.

TABLE n. 4
Quali-quantitative compositions of Nasal Compositions of Insulin (human
recombinant) nasal spray solution to be tested.

| 1.0 ml of nasal spray solution | NASAL COMPOSITION N^{o}. | | | | | |
|---|---|---|---|---|---|---|
| containing: | 7 | 8 | 9 | 10 | 11 | 12 |
| Peptide and ingredients: | mg | mg | mg | mg | mg | mg |
| Insulin human recombinant (U) | 500 U | | | | | |
| THAM | 1.5 | 3.0 | 5.8 | 12.0 | 24.0 | 30.0 |

| | |
|---|---|
| Hydrochloric acid | 2.9 |
| Methyl p-hydroxybenzoate | 1.2 |
| Distilled water q.s. to ml | 1.00 |

### Subjects and Methods

Administered single dose of 50 U of insulin per subject, study design (6 healthy subjects), blood sampling and analytical procedures were carried out with applicable methods as those described in the published literature (2) (3) (4). Each insulin Nasal Composition (as spray solution) was individually administered by intranasal administration route by means of a device (dosing pump and nasal applicator), such as those commonly available on the market. Each actuation volume is dispensing a volume of 0.10 ml of nasal solution, corresponding to 50 U of insulin human recombinant.

### Pharmacodynamic and Pharmacokinetic Calculations

For each tested Nasal Composition from NO. 7 to 12, a blood sample was collected at 20 minutes after a single dose (50 U) administration to each subject by nasal route, being the Maximum level of insulin plasma concentration reached in about 20 minutes, as described in the published literature (2). The individual insulin Maximum plasma concentrations and the mean (arithmetic mean and S.D.) plasma concentrations are reported in Table n. 5.

TABLE n. 5
Individual insulin human recombinant Maximum plasma
concentrations and arithmetic mean and S.D.

| Insulin Maximum plasma | NASAL COMPOSITION N^{o}. | | | | | |
|---|---|---|---|---|---|---|
| concentrations (μU/ml) | 7 | 8 | 9 | 10 | 11 | 12 |
| Subject 5 | 12.3 | 19.1 | 53.7 | 43.8 | 36.9 | 29.1 |
| Subject 6 | 25.4 | 29.4 | 53.2 | 51.1 | 44.5 | 35.7 |
| Subject 7 | 14.3 | 17.8 | 38.3 | 39.4 | 33.6 | 41.2 |
| Subject 8 | 24.7 | 23.0 | 46.7 | 52.3 | 45.6 | 36.1 |
| Subject 9 | 19.6 | 28.9 | 54.1 | 41.7 | 32.3 | 44.8 |
| Subject 10 | 24.5 | 18.6 | 44.5 | 50.6 | 41.8 | 34.9 |
| Mean | 20.1 | 22.8 | 48.4 | 46.5 | 39.1 | 36.9 |
| S.D.± | 5.72 | 5.24 | 6.38 | 5.52 | 5.66 | 5.44 |

TABLE n. 6
Arithmetic mean of Maximum plasma insulin concentrations
(μU/ml).

| | NASAL COMPOSITION N^{o}. | | | | | |
|---|---|---|---|---|---|---|
| PARAMETERS | 1 | 2 | 3 | 4 | 5 | 6 |
| THAM concentrations | 1.5 | 3.0 | 5.8 | 12.0 | 24.0 | 30.0 |
| (mg/ml) | | | | | | |
| Arithmetic mean | 20.1 | 22.8 | 48.4 | 46.5 | 39.1 | 36.9 |
| Maximum plasma | | | | | | |
| concentrations of Insulin | | | | | | |
| (μU/ml) | | | | | | |

### Discussion and Conclusion

In this study the absorbefacient effect of THAM in pharmaceutical Nasal Compositions containing insulin (human recombinant) has been investigated in relation to the absorption rate of the peptide.

The experimental conditions and determinations of the case study have been carried out by following, when applicable, the procedures, such as those described in published literature (2) (3).

The Maximum insulin plasma concentrations (1 U/ml) have been determined at the predictable maximum peak of 20 minutes following to a single dose nasal administration of 50 U of each Nasal Composition under testing.

The results of the above study, demonstrate the enhanced absorbefacient effect of THAM on the peptide insulin (human recombinant) contained in Nasal Compositions.

While the various embodiments of the present invention have been described herein, it is possible that one skilled artisan could modify the combination of the basic and optional ingredients and the production conditions and obtain similar or equivalent results. Such modifications are contemplated as being within the scope of the present disclosure.

What is claimed is:

1. A pharmaceutical composition comprising:
(1) a therapeutically effective amount of a pharmaceutically active nasal peptide or its pharmaceutically acceptable salt or its peptidic fragment and
(2) an absorbefacient effective amount of THAM in a pharmaceutically acceptable, aqueous liquid diluent or carrier, said composition being in a convenient form for nasal administration.

2. A nasally administrable pharmaceutical composition, according to claim 1, wherein the nasal peptide, its pharmaceutically acceptable salt or its peptidic fragment is selected from the group consisting of the physiologically active lymphokines and monokines, peptidic enzymes, proteic vaccines, peptidic toxoids and personalized proteins derived from genoma, in a form suitable for nasal administration.

3. A nasally administrable pharmaceutical composition, according to claim 1, wherein the therapeutically affective amount of nasal peptide, its pharmaceutically acceptable salt or its peptidic fragment is selected from the group consisting of the peptide hormones and hormone antagonists buserelin, desmopressin, vasopressin, angiotensin, felypressin, octreotide, somatropin, thyrotropin (TSH), somatostatin, gosereline, thryptorelin and insulin selected from the group consisting of caw and pig, synthetic and recombinant.

4. A nasally administrable pharmaceutical composition, according to claim 1, wherein the therapeutically affective amount of nasal peptide, its pharmaceutically acceptable salt or its peptidic fragment is further selected from the group consisting of the peptide hormones and the hormone derivatives protirelin, adrenocorticotropin (ACTH), prolactin, luteinizing hormone (LH), luteinizing hormone-release hormone (LH-RH), leuprorelin, calcitonins, carbocalcitonin and calcitonin gene related peptides (CGRP).

5. A nasally administrable pharmaceutical composition, according to claim 1, wherein the therapeutically affective amount of nasal peptide, its pharmaceutically acceptable salt or its peptidic fragment is further selected from the group consisting of the peptide hormones and the hormone derivatives kallikrein, parathyrin, glucagon, oxytocin, gastrin, secretin, leptin, nafarelin, serum gonadotropin, gonadotropin release factor, growth hormone, erytropoietin, hirudin, urograstrone, rennin, human parathyroid hormone (h-PTH) and teriparatide [h-PTH(1-34)].

6. A nasally administrable pharmaceutical composition, according to claim 1, wherein the therapeutically affective amount of nasal peptide, its pharmaceutically acceptable salt or its peptidic fragment is further selected from the group consisting of physiologically active lymphokines and monokines interferon, interleukin, transferrin, histaglobulin, macrocortine, endorphins, enkephalins and neurotensin.

7. A nasally administrable pharmaceutical composition, according to claim 1, wherein the therapeutically affective amount of nasal peptide, its pharmaceutically acceptable salt or its peptidic fragment is further selected from the group consisting of the peptidic enzymes native human lysozyme, urokinase and superoxide dismutase.

8. A nasally administrable pharmaceutical composition, according to claim 1, wherein the therapeutically affective amount of nasal peptide, its pharmaceutically acceptable salt or its peptidic fragment is selected from the group consisting of the proteic vaccines acellular and cellular pertussis, diphtheria, tetanus and influenza vaccines.

9. A nasally administrable pharmaceutical composition, according to claim 1, wherein the therapeutically affective amount of nasal peptide, its pharmaceutically acceptable salt or its fragment is further selected from the group consisting of the peptidic toxoids diphtheria, tetanus and from the group of personalized proteins derived from genoma.

10. A nasally administrable pharmaceutical composition, according to claim 1, wherein the therapeutically effective amount of pharmaceutically active nasal peptide, its pharmaceutically acceptable salt or its peptidic fragment is in a concentration of 0.001 microgram/ml to 50.0 mg/ml or of 10 Units/ml to 20000 Units/ml, in relation to the therapeutically effective dose for administration by the endonasal route and the absorbefacient effective amount of THAM is in a concentration of 4.5 mg/ml to 30.0 mg/ml.

11. A nasally administrable pharmaceutical composition, according to claim 1, wherein the therapeutically effective amount of pharmaceutically active nasal peptide, its pharmaceutically acceptable salt or its peptidic fragment is in a concentration of 0.01 microgram/ml to 50.0 mg/ml or of 20 Units/ml to 12500 Units/ml and the absorbefacient effective amount of THAM is in a concentration of 4.5 mg/ml to 10.0 mg/ml.

12. A nasally administrable pharmaceutical composition, according to claim 1, wherein the therapeutically effective amount of active nasal peptide, its pharmaceutically acceptable salt or its peptidic fragment is in a concentration of 0.05 microgram/ml to 10.0 mg/ml or of 100 Units/ml to 6000 Units/ml and the absorbefacient effective amount of THAM is in a concentration of 4.6 mg/ml to 5.0 mg/ml.

13. A nasally administrable pharmaceutical composition, according to claim 1, wherein said pharmaceutical composition is in the form of ready-to-use aqueous solution suitable for nasal administration in the form of a nasal spray.

14. A nasally administrable pharmaceutical composition, according to claim 1, for administration in single dose volume or in multiple dose thereof, each actuation comprising a metered dose volume between 50 microlitres and 200 microlitres.

15. A nasally administrable pharmaceutical composition, according to claim 1, wherein the pharmaceutically acceptable aqueous carrier further comprises the pharmaceutically acceptable auxiliary additive hydrochloric acid or citric acid and one or a mixture of methyl or/and propyl p-hydroxybenzoate and/or cysteine.

16. The method according to claim 15, wherein the pharmaceutically acceptable aqueous carrier further comprises the pharmaceutically acceptable additive hydrochloric acid 0.1 N in a concentration of 0.3 mg/ml to 50.0 mg/ml or citric acid in a concentration of 0.6 mg/ml to 60.0 mg/ml and one or a mixture of methyl or/and propyl p-hydroxybenzoate in a concentration not exceeding 0.3 mg/ml with a ratio of 2:1 to 20:1 and cysteine in a concentration of 0.5 mg/ml to 10.0 mg/ml.

17. A method for treating a patient which comprises intranasally administering in the form of nasal spray to said patient, a dosed volume of a composition according to claim 1 to elicit the desired pharmacological effect.

18. The method, according to claim 17, in which the administrable dose volume of a nasally administrable pharmaceutical composition, comprised in a metered monodose disposable or in a multidose system thereof, is comprised between 50 microliters and 200 microliters per actuation.
